(19) United States
(12) Patent Application Publication
(10) Pub. No.: US 2022/0233433 A1
(43) Pub. Date: Jul. 28, 2022

(54) Device in the Form of a Cannabinoid-Based Polymer Matrix

(71) Applicant: AB7 INNOVATION SASU, DEYME (FR)

(72) Inventors: Arnaud VILBERT, BAZIEGE (FR); Sophie LECLERC, ESCALQUENS (FR)

(21) Appl. No.: 17/617,470

(22) PCT Filed: Jun. 25, 2020
(86) PCT No.: PCT/FR2020/000194

§ 371 Date: Dec. 8, 2021

Related U.S. Application Data
(60) Provisional application No. 63/016,374, filed on Apr. 28, 2020.
(60) Provisional application No. 62/866,253, filed on Jun. 25, 2019.

(51) Int. Cl. A61K 9/00; A61K 47/32; A61K 31/05; A61K 36/534; A61P 29/00; A61K 31/045; A61K 36/53; A61K 47/24; A61K 47/14; A61K 36/61; A61K 47/34; A01K 27/00; A44C 5/00

(52) U.S. Cl. CPC A61K 9/0017; A61K 47/32; A61K 31/05; A61K 36/534; A61P 29/00; A61K 31/045; A44C 5/0007; A61K 47/24; A61K 47/14; A61K 36/61; A61K 47/34; A61K 9/0014; A01K 27/007; A61K 36/53

(57) ABSTRACT

The invention relates to a device for topical application in the form of a polymer matrix containing at least one active ingredient from the family of cannabinoids, alone or as a mixture with other active ingredients. The invention preferably relates to the device in the form of a collar, to the use thereof for improving the well-being and the mobility of an animal to which the device is applied, and to a process for producing the device.

The present invention is in the field of treatments for the relief of different symptoms linked to pain or stress. More specifically, the present invention relates to a device for topical application in the form of a polymer matrix containing at least one active ingredient from the family of cannabinoids, alone or as a mixture with other active ingredients. The invention preferably relates to the device in the form of a collar, to the use thereof for improving the well-being and the mobility of an animal to which the device is applied, and to a process for producing the device.

The use of cannabis in medicine has been known since antiquity. The numerous therapeutic effects of cannabis known at present are due to the psychoactive molecules present in cannabis, cannabinoids, and in particular cannabinol (CBN), delta-9-tetrahydrocannabinol (THC) or cannabidiol (CBD). THC is mainly responsible for the euphoric, psychotropic properties and gives cannabis the status of a drug. Only very recently has the administration of cannabidiol (CBD), one of the major active compounds of cannabis (also known as hemp), been commercialized. Thus, varieties of hemp with no narcotic properties (with no THC) may be used for industrial and commercial ends subject to three cumulative conditions:
the authorized varieties of hemp are listed in the French Code of Public Health,
only the seeds and fibers may be used. The use of the flowers, however, is prohibited,
the plant must have a content of less than 0.2% of delta-9-tetrahydrocannabinol (THC),
CBD-based products are prohibited if they contain THC, in whatever quantity.

Said THC-free cannabinoids have been used since antiquity for the improvement in quality of life they bring to people and animals affected by situations such as pain, stress, anxiety, epilepsy, spasms, arthritis, nausea or dermatological problems. Said cannabinoids were basically administered orally or sublingually. For topical administration, cannabidiol oils are formulated either in semi-solid form (ointments, creams, gels) or in liquid form (milks, emulsions, lotions). The above-mentioned galenics for topical application require repeated applications in order to produce the systemic effects expected. Moreover, in the veterinary field, the application of such topical products is neither easy nor optimal to obtain effective penetration through the fur of the animal, or for long-term treatment.

One of the objects of the invention is to propose a more advantageous topical administration method for localized treatment of pain or stress while limiting the known systemic secondary effects of the oral method and that can be used for more long-term treatment. In the field with which the invention is concerned, when a composition is applied topically, it is observed that a portion of the constituents may disappear owing to their volatility, oxidative degradation, UV exposure or through washing. This result is that at the very most 40% of the available effective dose of active ingredients passes through. The bioavailability of the active ingredients is not optimal and consequently the effectiveness thereof in relieving pain or stress is reduced.

To overcome the various drawbacks mentioned above and to improve the passage of the active ingredients through the skin, the applicant proposes the use of a solid polymer matrix as a vehicle for the topical and/or transdermal application of cannabinoids, alone or in combination with other active ingredients, said matrix allowing a composition having stable incorporated active ingredients to be obtained. Stable according to the invention should be understood as the physical stability of the device and the chemical stability of the active ingredients in the device. Physical stability means the absence of exudation confirming that the active phase is correctly incorporated, the mechanical strength of the device, particularly resistance to deformation, flexibility or hardness required for the end use. Chemical stability means the absence of degradation of the main active ingredient or ingredients, and that said degradation of the active ingredient should be less than 10% of the amount introduced initially in the device. The device according to the invention should be stable to evaporation and to the risk of loss, particularly through leaching, offering in use continuous, controlled, effective and gradual release of the active ingredients on the subject being treated.

One of the objects of the present invention is also to propose a device for which the manufacturing method is easy to implement and can be transposed to an industrial scale. The prior art in the field of polymers describes the difficulties of incorporating active ingredients in said polymers. This is because it must be possible to incorporate the active ingredient completely and stably in the polymer, avoiding uncontrolled release or delivery. In response to these problems, the prior art describes various solutions such as the addition of numerous specific ingredients to make incorporation of the active ingredient possible, or proposing the incorporation of the active ingredient in particulate structures that are to varying degrees complex to produce. The method for obtaining the device according to the invention allows the incorporation of liquid active ingredients within the structure polymer of the matrix to be obtained without having to use specific ingredients or incorporate the active ingredient in particulate form.

The device according to the present invention may be defined as for topical application, as said device is for local use applied to the skin or coat of the subject to be treated. The device of the invention may also meet the definition by a person skilled in the art of a transdermal device, being formed of a polymer matrix used as a support for the active ingredients and produced in order to allow said active ingredients to be released for delivery to the subject to be treated so that the active ingredients are able to reach the cannabinoid receptors. In the rest of this application, the terms “topical” and “transdermal” will be applied without distinction to the device according to the present invention.

The transdermal systems containing the cannabinoid derivatives are described in the prior art. However, these all describe systems of the reservoir or matrix type, made up of at least two, or even a plurality of layers of different materials having diverse functions. Said transdermal systems are made up of at least one adhesive layer to attach the device to the subject, a reservoir layer containing the active ingredient or ingredients to be conveyed and an impermeable outer layer. The prior art does not describe a non-adhesive single-layer system that allows a cannabinoid active ingredient to be easily incorporated and delivered, as proposed in the present invention. Moreover, the systems described are complex to produce and not suitable for use on an animal subject covered with fur.

In the present invention, the applicant therefore proposes the use of at least one cannabinoid, alone or in association with other active ingredients as active analgesic or anti-stress agents within a single-layer, monopolymeric polymer matrix that has never been described in the prior art. A single-layer polymer matrix according to the present invention should be understood as a matrix made up of a single layer of polymer and not the juxtaposition of a plurality of layers as commonly described in the prior art. Monopolymeric according to the present invention should be understood as a matrix made up of the same polymer. Said same polymer for its part may be a homopolymer or a copolymer. A homopolymer is a polymer originating from a single monomer species (real, implicit or hypothetical). All the repeating units of a homopolymer are of the same chemical nature. A copolymer is a polymer originating from at least two different monomers, known as comonomers; it contains at least two types of repeating units.

### BRIEF DESCRIPTION OF THE FIGURES

FIG. 1 shows the CBD release kinetics from a device according to an embodiment of the invention according to Example 10.

FIG. 2 shows the CBD release kinetics from devices according to embodiments of the invention according to Examples 5 and 11.

Surprisingly, it was found that by incorporating a cannabinoid and more particularly cannabidiol (CBD) in the form of an isolate or an oil rich in cannabinoids derived from hemp as the only active ingredient or preferably in combination with essential oils in a solid polymer matrix as described in the present invention, the result is a stable composition and effective relief from the pain or stress which is manifested in the human or animal subject treated by means of the device according to the invention. There is therefore a need for the present invention which helps relieve pain or stress and allows the stability of the composition to be increased while ensuring the bioavailability of said constituents to obtain a more effective analgesic or stress-relieving effect and an extended period of effectiveness.

Consequently, a first object of the present invention consists of a device comprising a solid polymer matrix incorporating a composition of active ingredients containing at least a first active ingredient formed by a cannabinoid in the form of an oil or an isolate of hemp extract or cannabis, alone or in association with other active ingredients. The active ingredients used in association with the cannabinoid active ingredient according to the present invention may be chosen from analgesics, anti-inflammatories, soothing or anesthetic active ingredients. According to one embodiment, the cannabinoid active ingredients may also be associated with insecticidal or pest-repellent active ingredients.

According to the present invention, the cannabinoids used are preferably cannabidiol (CBD) which may be used in the form of a powder, liquid, solution, oil or isolate, or hemp plant extract. CBD oil is understood preferably as an oil rich in cannabinoids, more particularly rich in phytocannabinoids and terpenes identified in hemp genes. Said oils are in particular oils originating from Cannabis sativa, Cannabis indica and Cannabis ruderalis plants. Preferably, the CBD oil used contains between 50% and 95% of cannabidiol (CBD) and preferably between 80% and 90% of CBD in addition to various terpenes. In a particular embodiment where specific medical treatments are indicated, the CBD oil used according to the present invention may contain THC (also known as full spectrum CBD). However, in a preferred embodiment according to the invention, the oil used is an oil known as a broad-spectrum oil, of which the THC content is less than 0.2%, preferably free of THC and which still helps relieve pain or stress or any other related pathology. In the rest of the text, the terms “CBD oil” or “CBD-rich hemp oil” will be used indiscriminately to refer to CBD in the form of an oil extracted from hemp rich in cannabidiol (CBD), and of which the THC content is less than 0.2% p/p. In the composition according to the invention, the CBD oil is present in an amount of between 0.01% and 10% p/p, preferably between 0.1% and 5% by total weight of the device. The cannabidiol and terpene purification and extraction system may in particular but not exclusively be based on a patented technology which preserves the terpenes and lipids of the plant while being free of undesirable compounds such as delta-9-THC, waxes and chlorophyll, as described in particular in patent application US2018/0333446 incorporated here by reference in its totality.

In a particularly preferred way, the CBD used is in the form of an isolate. Isolate according to the present invention should be understood in particular as a crystalline powder obtained by extraction from the hemp plant, with a CBD concentration greater than or equal to 99%. In a preferred embodiment, the CBD isolate used in the composition is present in an amount of between 0.01% and 10% p/p, preferably between 0.1% and 5% p/p, and more preferably between 0.5% and 2.5% p/p of the composition.

In a preferred embodiment according to the invention, the active ingredients that may be used in combination with the cannabinoids are essential oils, or monoterpenic alcohol, aldehyde or ester derivatives of essential oils. Preferably, said active ingredients are chosen from linalool, menthol or essential oils of peppermint, lavender, lavandin, Italian helichrysum, cedar, lemon, citronella, carrot, ginger, niaouli, sweet orange, clove, Eucalyptus citriodora, Eucalyptus radiata, geranium, valerian, Nepteta cataria, chamomile, ylang ylang, clary sage, mandarin, sandalwood, bergamot, marjoram, frankincense, geranium, thyme, juniper or mixtures thereof. Preferably, the CBD oil or CDB isolate will be associated with menthol, linalool, essential oil of peppermint, Eucalyptus citriodora, lavender, lavandin, valerian or Nepeta cataria. More preferably, in the soothing device according to the invention, the active ingredients are CBD isolate or CBD oil and essential oil of peppermint or Eucalyptus citriodora for the soothing and/or analgesic action thereof coupled with that of CBD. In the device according to the invention, the active ingredients in the form of essential oils or compounds of essential oils are present in an amount of between 0.5% and 20% p/p, preferably between 0.5% and 15% p/p, and more preferably between 0.5% and 6% by total weight of the device.

According to a first variant, the polymer matrix is made up of thermosetting or thermoset polymers, chosen from cast polyurethane, reactive silicones and epoxy resins, preferably cast polyurethane. The cast polyurethane matrix according to the present invention is described in patent FR2992325 incorporated here by reference in its totality.

According to a second preferred variant of the device according to the invention, the polymers forming the solid matrix are chosen from thermoplastic polymers, which may or may not be biodegradable, chosen from the group made up of the polyolefins and derivatives thereof, ethylene butyl acrylates, polyamides, copolyamides and derivatives thereof, polyvinyl chlorides (PVC), polyurethanes (TPU) and derivatives thereof, styrenes and derivatives thereof, vulcanized thermoplastics, agro-polymers and derivatives thereof, polyesters and derivatives thereof, taken alone or as a mixture. The polyolefins and derivatives thereof may in particular be chosen from the polyethylenes (PE), polypropylenes (PP), ethylene and vinyl acetate copolymers (EVA). The styrenes and derivatives thereof may be chosen in particular from the polystyrene-poly(ethylene butylene)-polystyrene copolymers (SEBS), polystyrene-polyisoprene-polystyrene copolymers (SIS) or polystyrene-polybutadiene-polystyrene copolymers (SBS). The copolyamides and derivatives thereof may in particular be chosen from the ether-block-amides (EBA). The agro-polymers and derivatives thereof may in particular be chosen from the polysaccharides, starch, cellulose and derivatives or proteins.

Preferably, the thermoplastic polymer that may be used according to the invention is chosen from a polyolefin and derivatives thereof or a polyurethane and derivatives thereof. More preferably, the polymer matrix forming the device according to the present invention is a thermoplastic made up of ethylene and vinyl acetate copolymers (EVA) or a polyether or polyester unit polyurethane (TPU). The applicant has demonstrated that these two preferred polymers would confer optimal loading and release properties for the active ingredients. Said two polymers allow both the mechanical properties of elasticity and hardness sought for use in the device and optimal incorporation and release capacities of the active ingredients using a method that is easily implemented on an industrial scale. Preferably, for a device with a transparent, translucent appearance, TPU will be preferred. The polymer represents between 40% and 99% by weight of the device. Preferably the polymer matrix is in a sufficient amount to make up 100% (qs 100%) of the composition of the device.

According to a particularly preferred embodiment, if the device is a collar for an animal, the thermoplastic polymer that may be used according to the invention is non-adhesive. This is because an adhesive device would not be suitable for application to the fur of the animal.

According to a particularly preferred embodiment, to meet the object of maintaining the stability and integrity of the device for the long-term treatment of the subject, the thermoplastic polymer that may be used according to the invention is not hydrosoluble.

Moreover, unlike the polymer matrices found in the prior art formed of a polymer mixture and/or a plurality of layers or sequences of polymers, it is specified that the matrix forming the device according to the invention is solely a single-layer, monopolymeric matrix, and is not formed by an association of a plurality of sequences of different polymers.

In the present invention, the person skilled in the art may add other additives to the formulation of the device. As non-exhaustive examples, the formulation additives may be dispersion agents, solvents, vectorizers, pro-penetrants, plasticizers, stabilizers, colorants or perfumes for adding to the composition according to the invention depending on the effect sought.

As examples of dispersion agents, solvents, vectorizers or pro-penetrators, oils of organic, synthetic, mineral or plant origin, esters such as mixtures of succinate, glutarate and adipate, oils such as omega 3 polyunsaturated fatty acid, fatty acid esters, glycol ethers, or squalene and derivatives thereof may be cited.

A preferred embodiment of the device according to the invention comprises vectorizers or pro-penetrants that may facilitate the vectorization of the active ingredient on the skin or fur of the subject and/or the transcutaneous passage of the active ingredients. In a preferred embodiment, said vectorizers or pro-penetrants are lipophilic compounds. As an indication, coconut oil, jojoba oil, hemp oil, macadamia oil, linseed oil, sweet almond oil, krill oil, diethylene glycol monoethyl ether, marketed under the name Transcutol V, isopropyl myristate, or derivatives of squalene, preferably 2,6,10,15,19,23-hexamethyltetracosane, also known as perhydrosqualene or dodecahydrosqualene marketed under the name Squalane, may be cited. Said ingredients will be present in an amount of between 0.1% and 30% p/p, preferably between 0.5% and 15% p/p, more preferably between 1% and 6% by total weight of the device.

According to the present invention, the device also comprises a plasticizing compound. This is because the applicant noted surprisingly that the plasticizer had a role in the release of the active ingredients from the polymer matrix, and that the nature and concentration of plasticizer allowed the release of the active ingredients to be modulated. As a plasticizer that may be used in the device according to the invention, ethylhexyl diphenyl phosphate, dioctyl adipate, diisooctyl adipate, dibutyl adipate, dimethyl adipate, dimethyl glutarate or dimethyl succinate may be cited, taken alone or as a mixture. According to a particular embodiment the plasticizer mixture composed of dimethyl adipate, dimethyl glutarate and dimethyl succinate is used in the form of a commercial mixture known as Coasol. Preferably, the plasticizers will be different from phthalates, the use of which is controversial owing to their toxicity. In a preferred embodiment according to the invention, the plasticizer or the plasticizer mixture is present in an amount of between 0% and 50% by weight, preferably between 5% and 35% by total weight of the composition. In addition, the plasticizer allows the plastic properties sought for the use thereof to be conferred on the matrix. According to the present invention, the plasticizer also has a role as a softener of the polymer materials used to give the device the flexibility or hardness chosen. This is because, as a preferred embodiment, one of the objects of the present invention is a device in the form of a collar for an animal. Said collar therefore requires sufficient flexibility to be adapted to the shape of the neck of the animal while retaining the required shape and structure and the active ingredient release properties sought. According to a particularly preferred embodiment according to the invention, in particular if the polymer used is TPU, the polymer matrix according to the invention comprises at least one plasticizer.

In a particular embodiment, stabilizers and/or antioxidants may be added to the composition of the device according to the invention at a concentration of between 0% and 5% p/p, preferably between 0% and 1.5% by total weight of the composition. As a preferred example, vitamin E or tocopherol may be cited, which will be present at between 0.5% and 1.5% by total weight of the composition.

Colorants as well as perfumes may also be added to the composition. The colorants may be present in liquid or solid form. As an example of a perfume, said perfume is chosen from vanilla, green lemon, lavender, violet, apple, apricot, patchouli or green bamboo leaf and may be adapted to the indication and to the user.

A person skilled in the art will ensure that the formulation additives are chosen so as not to interfere with the properties sought for the composition according to the invention.

According to a variant, the device according to the invention comprises as % by weight of the device:
between 0.01% and 10% of CBD isolate or CBD oil,
between 0.5% and 20% of complementary active ingredients,
between 0.1% and 30% of vectorizing additives,
between 0% and 50% of plasticizing additives,
qs 100% by weight of solid polymer matrix, chosen from EVA or TPU.

According to a particular embodiment, the device according to the invention comprises, as % by weight of the device:
between 0.01% and 10% of CBD isolate or CBD oil,
between 0.5% and 20% of complementary active ingredients,
between 0.5 and 15% of vectorizing additives,
between 0% and 1.5% of antioxidant additives,
qs 100% by weight of solid EVA polymer matrix.

According to another particular embodiment, the device according to the invention comprises as % by weight of the device:
between 0.1% and 5% of CBD isolate,
between 0.5% and 15% by weight of active ingredients in the form of essential oil and/or essential oil compounds,
between 0.5% and 15% of vectorizing additives,
between 5% and 35% of plasticizing additives,
qs 100% by weight of TPU solid polymer matrix.

According to a more preferred embodiment, the device according to the invention comprises as % by weight of the device:
between 0.1% and 5% of CBD isolate,
between 0.5% and 15% by weight of active ingredients in the form of essential oil and/or essential oil compounds,
between 1% and 6% of vectorizing additives,
between 5% and 35% of plasticizing additives,
qs 100% by weight of TPU solid polymer matrix.

According to a second more preferred embodiment, the device according to the invention comprises as % by weight of the device:
between 0.1% and 5% of CBD isolate,
between 0.5% and 15% by weight of active ingredients in the form of essential oil and/or essential oil compounds, more preferably peppermint essential oil,
between 1% and 6% of vectorizing additives, more preferably squalane,
between 5% and 35% of plasticizers, preferably a mixture of ethylhexyl diphenyl phosphate, dimethyl adipate, dimethyl glutarate and dimethyl succinate or dimethyl glutarate,
qs 100% by weight of solid polymer matrix, composed of TPU.

According to one embodiment, the device is presented in the form of a collar, bracelet, medallion, single-layer patch or textile coating. Preferably, the device is produced in the form of a collar or bracelet. In a preferred embodiment, the method for obtaining the device is an injection molding method. In a particularly preferred embodiment, the device is a collar for a pet, in particular a collar for a dog or cat.

A second object of the present invention is a device consisting of a solid polymer matrix incorporating a solution of active ingredients containing at least one CBD isolate or CBD oil for use in the treatment or relief of pain or stress in a human or animal subject. More particularly, having regard for the properties of CBD in combination with the other active ingredient or ingredients according to the invention, applying the device allows pain caused in particular by inflammatory disorders, whether immune-induced or not, to be regulated. As an example, the device allows cutaneous pain or pruritus, muscular pain, joint pain, tendinitis and rheumatic pain such as arthrosis or arthritis to be regulated in a human or animal subject. The device according to the invention may also be used to regulate stress in a human or animal subject.

The present invention therefore relates to the device for topical/transdermal application in a thermoplastic polymer matrix loaded with active ingredients, in particular cannabidiol, for use in the relief of pain or stress in a human or animal subject to be treated. Said device is characterized in that it is placed in contact with the skin and/or fur. According to a preferred embodiment, said device helps ensure the relief of pain or stress in an animal, characterized in that it is applied in contact with the skin and/or fur in the form of a collar. The active ingredients are thus released from the collar and by topical application penetrate the skin, transported in particular by the sebum. The present invention therefore also relates to the device for use to improve the mobility and well-being of a subject and more particularly of an animal.

Another object of the present invention is the device according to the invention for use to administer cannabidiol, and any other active ingredient incorporated in the device, transdermally. This delivery route thus offers the advantage of reducing any metabolic effect conventionally obtained during the oral administration of the active ingredient and increasing the bioavailability of the active compounds. Moreover, the use of the transdermal device according to the invention has the advantage of delivering the active ingredient gradually, continuously and in a measured way, and over a longer period of time. The application time required for the devices according to the invention is from 30 minutes for a rapid effect to several months for a long-term effect. According to a preferred embodiment, the device according to the invention is applied for a period of 1 day to 60 days, preferably for 28 days (four weeks). This is because according to a preferred embodiment said time of four weeks is necessary and sufficient to treat a human or animal subject for pain or stress before a possible new application, if necessary.

Another object of the present invention is a method of manufacturing the device according to the invention. This is because, surprisingly, the choice of the process, and in particular the mixing temperatures, makes it possible to carry out a manufacturing process in a few steps without having recourse to the addition of specific ingredients or a particulate structure to assist in the incorporation of the active ingredients in the polymer matrix.

According to the present invention, the method for manufacturing the device according to the invention comprises the following four main steps: heating the polymer, preparing the active phase, mixing the active phase and the polymer, shaping the device.

The first step of the method therefore consists of placing the polymer in a reactor pre-heated to the required temperature depending on the nature of the polymer. In a preferred embodiment, the polymer will be heated to a temperature of between 10 and 15 degrees Celsius below its melting temperature, to a temperature sufficient to confer a structure on the polymer that allows the networks to open to facilitate the incorporation of the active ingredients without reaching the melting point of said polymer.

As an example, in a particular mode according to the invention when the polymers used are polyurethanes (TPU), the reactor will be heated to between 80 and 90 degrees Celsius, preferably between 90° C. and 95° C. In another particular embodiment according to the invention, when the polymers used are ethylene vinyl acetate copolymers (EVA), the reactor will be heated to between 45 and 90 degrees Celsius, preferably between 70° C. and 75° C. The reactors are then stirred.

The second step of the method consists of preparing the liquid active phase according to the following protocol: weighing and measuring the plasticizing and vectorizing ingredients and stirring to homogenize in order to obtain a limpid mixture. The active ingredients are then added while stirring at ambient temperature until a limpid, translucent and homogeneous mixture is obtained.

The third step of the method consists of gradually adding the active phase to the polymer until the target temperature selected for incorporation is reached. Stirring is maintained until the active ingredients are completely incorporated in the polymer. This is cooled to ambient temperature while stirring. The colorants or other additives are added to the reactor. The reactor is then emptied and the fourth and final injection molding step is then carried out to obtain the desired shape for the device according to injection molding methods known to persons skilled in the art.

Another object of the present invention is the product obtained by the method defined above.

To sum up, the invention relates to a device for topical application made up of a single-layer polymer matrix characterized in that the polymer is chosen from the group consisting of an ethylene vinyl acetate copolymer and a polyurethane, said matrix also being characterized in that it comprises at least one cannabinoid derivative and between 0.5% and 20% by total weight of the matrix of at least one essential oil or monoterpenic derivative of essential oils.

The invention relates more particularly to a device characterized in that the polymer is non-adhesive.

The invention relates more particularly to a device characterized in that the polymer is an ethylene vinyl acetate copolymer or a polyurethane.

The invention relates to said device, characterized in that the cannabinoid derivative is cannabidiol, preferably free of delta-9-tetrahydrocannabinol. More preferably, said device is characterized in that the cannabidiol is present in the form of a CBD isolate or a CBD oil, said CBD isolate contains at least 90% CBD and said CBD oil comprises between 80% and 90% CBD.

The invention also relates to the above device characterized in that it also comprises at least one essential oil or monoterpenic derivatives of essential oils, chosen from linalool, menthol or essential oils of lavender, lavandin, Italian helichrysum, cedar, lemon, citronella, carrot, ginger, niaouli, sweet orange, clove, Eucalyptus citriodora, Eucalyptus radiata, peppermint, geranium, valerian, Nepeta cataria, chamomile, ylang ylang, clary sage, mandarin, sandalwood, bergamot, marjoram, frankincense, geranium, thyme, juniper or mixtures thereof.

The device according to the present invention is also characterized in that it comprises a vectorizing compound chosen from oils of organic, synthetic, mineral or plant origin, esters such as mixtures of succinate, glutarate and adipate, oils such as omega 3 polyunsaturated fatty acid, fatty acid esters, glycol ethers, or squalene and derivatives thereof.

In the case of a particular polymer, and in particular if the polymer used is TPU, the device is characterized in that it also comprises a plasticizing compound chosen from ethylhexyl diphenyl phosphate, dioctyl adipate, diisooctyl adipate, dibutyl adipate, dimethyl adipate, dimethyl glutarate or dimethyl succinate, taken alone or as a mixture.

According to the present invention, the device is also characterized in that it is presented in the form of a collar, bracelet, patch, medallion or textile coating, preferably in the form of a collar.

More particularly, the invention relates to a device for topical application in a thermoplastic polymer matrix, loaded with active ingredients for use in the relief of pain or stress in an animal, characterized in that said device is placed in contact with the skin and/or fur, and in that the device comprises at least one cannabidiol isolate, free of delta-9-tetrahydrocannabinol, at least one essential oil or a compound of an essential oil and at least one non-adhesive thermoplastic polymer chosen from a polyurethane or an ethylene vinyl acetate copolymer.

The present invention also relates to the device defined above for use in the relief of stress or muscular, joint or rheumatic pain, such as tendinitis, arthritis or arthrosis.

The invention also relates to the use of a device according to the present invention characterized in that it allows the release and delivery of the cannabidiol to a human or animal subject topically or transdermally to be modulated.

The invention also relates to a manufacturing method for a device consisting of a thermoplastic polymer matrix comprising at least one non-adhesive thermoplastic polymer chosen from a polyurethane or an ethylene vinyl acetate copolymer, and in that the active ingredients comprise at least one cannabidiol isolate, free from delta-9-tetrahydrocannabinol or a cannabidiol oil free of delta-9-tetrahydrocannabinol and between 0.5% and 20% p/p of at least one essential oil or a compound of an essential oil, said method comprising the steps of heating the polymer to the chosen temperature, preparing the active phase, mixing the active phase with the polymer, then shaping the device, characterized in that said method does not comprise a step of adding specific excipients or particulate structures to aid in the incorporation of the active ingredients in the polymer matrix.

The invention also relates to the product obtained by the method described above.

The above examples serve to illustrate the invention without restricting the scope thereof. Also produced and defined in a particular shape (collar, bracelet, etc.), the compositions of the following examples may be adapted to different end forms. Only the final injection molding method will be modified and adapted depending on the final shape sought.

### Example 1: Analgesic Collar

Manufacture of a Collar as an Ethylene Vinyl Acetate Copolymer (EVA) Matrix Loaded with CBD-Rich Hemp Oil.

| Composition | Concentration (% p/p) |
|---|---|
| Transcutol V | 5 |
| CBD-rich hemp oil | 2 |
| Linseed oil | 3 |
| Peppermint oil | 0.75 |
| EVA | QS 100 |

The active ingredients, namely hemp oil rich in cannabinoid and peppermint oil, are placed in a beaker at ambient temperature. The linseed oil is added, the mixture is homogenized and then Transcutol V is added as a pro-penetrant. This is stirred gently using a bar magnet to obtain a homogeneous mixture which forms the analgesic active ingredient solution.
Next, a cylindrical mixer is pre-heated to 70° C. in a bath of oil. EVA pellets (Alcudia® PA-538) are placed therein, stirring gently, until the temperature measured in said pellets is close to 70° C. Next the previously obtained solution is placed in the mixer, still stirring gently. This is left stirring until all the liquid is completely absorbed by the polymer, then the temperature is lowered to 25° C. The mixer is emptied and the compound obtained in this way is stored in hermetically sealed packaging impervious to air and humidity. “Compound” refers to the term well known to a person skilled in the art to designate a polymer loaded with an active ingredient. The polymer loaded with active ingredients obtained above is injected as a collar weighing about 41 g at the nozzle outlet for a length of 75 cm. Said collar is intended to be worn by a dog around the neck to relieve various arthrosis-type pains.

### Example 2: Analgesic Collar

| Composition | Concentration (% p/p) |
|---|---|
| CBD-rich hemp oil | 1 |
| Isopropyl myristate | 1.5 |
| Linalool | 3 |
| Menthol | 2.5 |
| Linseed oil | 2 |
| EVA | QS 100 |

The three active ingredients, namely the hemp oil, linalool and menthol, are placed one after the other in a beaker at ambient temperature. Refined linseed oil is added to better disperse the active ingredients, then isopropyl myristate as a pro-penetrant. This is stirred gently using a bar magnet to obtain a homogeneous mixture which forms the solution of analgesic active ingredients.
Next, a cylindrical mixer is pre-heated to 70° C. in a bath of oil. The EVA pellets (Alcudia® PA-538) are introduced while stirring gently until the temperature measured in said pellets is close to 70° C. Next, the previously obtained solution is placed in the mixer, still stirring gently. This is left stirring until all the liquid is completely absorbed by the polymer, then the temperature is lowered to 25° C. The mixer is emptied and the compound thus obtained is stored in a hermetically sealed package impervious to air and humidity.
The polymer loaded with active ingredients obtained above is injected as a collar weighing 41 g at the nozzle outlet for a length of 75 cm. Said collar is intended to be worn around the neck by a dog to relieve various arthrosis-type pains.

### Example 3: Anti-Stress Medallion

| Composition | Concentration (% p/p) |
|---|---|
| Transcutol V | 3 |
| CBD-rich hemp oil | 5 |
| Valerian essential oil | 2 |
| EVA | QS 100 |

The active ingredients, namely the hemp oil rich in cannabinoids and valerian essential oil, are placed in a beaker at ambient temperature, then Transcutol V is added as a pro-penetrant. This is stirred gently using a bar magnet to obtain a homogeneous mixture which forms the solution of relaxing, anti-stress active ingredients.
Next, a cylindrical mixer is pre-heated to 70° C. in a bath of oil. The EVA pellets (Alcudia® PA-538) are introduced while stirring gently until the temperature measured in said pellets is close to 70° C. Next, the previously obtained solution of active ingredients is placed in the mixer, still stirring gently. This is left stirring until all the liquid is completely absorbed by the polymer, then the temperature is lowered to 25° C. The mixer is emptied and the compound thus obtained is stored in a hermetically sealed package impervious to air and humidity.
The polymer loaded with active ingredients obtained above is injected as a medallion weighing 15 g at the nozzle outlet. Said medallion is intended to be worn as a collar by a dog to calm said dog in the case of circumstances liable to cause stress.

### Example 4: Analgesic Bracelet

| Composition | Concentration (% p/p) |
|---|---|
| Transcutol | 3 |
| CBD-rich hemp oil | 4 |
| Menthol | 1.5 |
| Refined macadamia oil | 2 |
| Lavandula angustifolia | 5.5 |
| essential oil | |
| EVA | QS 100 |

Example 4 is the preparation of an active monopolymeric matrix that can be injected in the form of a bracelet. The manufacturing method is similar to that of Examples 1 to 3 above.

### Example 5: Soothing Collar

| Composition | Concentration (% p/p) |
|---|---|
| Squalane | 1 |
| CBD isolate | 0.75 |
| Peppermint essential oil | 0.75 |
| Ethylhexyl diphenyl phosphate | 15 |
| Dimethyl adipate, dimethyl | 8.5 |
| glutarate and dimethyl succinate | |
| Colorant | 0.1 |
| TPU | QS 100 |

Place the polymer in a reactor that has been pre-heated to the required temperature depending on the type of polymer, in this case for TPU, to about 90° C. to 95° C., to a temperature sufficient to confer a structure on the polymer that allows the networks to open for incorporation therein of the active ingredients. Stir gently.
Prepare the liquid active phase according to the following protocol:
weigh and mix the plasticizing and vectorizing ingredients and stir to homogenize in order to obtain a limpid mixture,
while stirring at ambient temperature, add the CBD isolate as a first active ingredient and leave stirring until a limpid, translucent mixture is obtained,
add the co-active ingredient, in this case peppermint essential oil, and homogenize to obtain a homogeneous, translucent mixture.
   Once the polymer is at the target temperature, gradually add the active ingredient mixture prepared previously.
   Leave stirring until the active phase is completely incorporated in the polymer.
   Cool to ambient temperature, still stirring.
   Add the colorant and homogenize the mixture.
   Empty the reactor and carry out the injection molding steps to obtain the desired shape for the device.
   The examples according to the invention are tested for physical and chemical stability, as defined above in the application. The results for the present device are as follows:

Temperature stability: 54° C.

| | D 0 | D 14 |
|---|---|---|
| Physical stability | No exudation, good | No exudation, good |
| | mechanical strength | mechanical strength |
| Chemical stability | 100% | 95.5% |
| (% CBD active | | |
| ingredient versus | | |
| initial amount | | |
| incorporated) | | |

UV light

| | D 0 | D 7 |
|---|---|---|
| Physical stability | No exudation, good | No exudation, good |
| | mechanical strength | mechanical strength |

The results show that the device is stable and has allowed the required active ingredients, including CBD, to be incorporated effectively.

### Example 6: Soothing Bracelet

| Composition | Concentration (% p/p) |
|---|---|
| EVA | QS 100 |
| Squalane | 1 |
| Dimethyl adipate, dimethyl | 8.5 |
| glutarate and dimethyl succinate | |
| Peppermint | 0.75 |
| CBD isolate | 0.75 |
| Colorant | 0.1 |

Place the polymer in a reactor that has been pre-heated to the required temperature depending on the type of polymer, in this case for EVA, to about 70° C., to a temperature sufficient to confer a structure on the polymer that allows the networks to open for incorporation therein of the active ingredients. Stir gently.
Prepare the liquid active phase according to the following protocol:
weigh and mix the plasticizing and vectorizing ingredients and stir to homogenize in order to obtain a limpid mixture,
while stirring at ambient temperature, add the CBD isolate as a first active ingredient and leave stirring until a limpid, translucent mixture is obtained,
add the co-active ingredient, in this case peppermint essential oil, and homogenize to obtain a homogeneous, translucent mixture.
   Once the polymer is at the target temperature, gradually add the active ingredient mixture prepared previously.
   Leave stirring until the active phase is completely incorporated in the polymer.
   Cool to ambient temperature while stirring.
   Add the colorant and homogenize the mixture.
   Empty the reactor and carry out the injection molding steps to obtain the desired shape for the device.

### Example 7: Analgesic Bracelet

| Composition | Concentration (% p/p) |
|---|---|
| EVA (*) | QS 100 |
| Isopropyl myristate | 3 |
| Linalool | 2.5 |
| CBD-rich hemp oil | 0.9 |
| Ethylhexyl diphenyl phosphate | 7 |
| Colorant | 0.1 |

The manufacturing method is similar to that of Example 6 above.

### Example 8: Analgesic Bracelet

| Composition | Concentration (% p/p) |
|---|---|
| TPU | QS 100 |
| Isopropyl myristate | 3 |
| Linalool | 2.5 |
| CBD-rich hemp oil | 0.9 |
| Ethylhexyl diphenyl phosphate | 17 |
| Colorant | 0.2 |

The manufacturing method is similar to that of Example 5 above.

### Example 9: Soothing Collar

| Composition | Concentration (% p/p) |
|---|---|
| EVA(*) | QS 100 |
| Isopropyl myristate | 3.00 |
| Eucalyptus citriodora | 0.75 |
| Peppermint | 0.40 |
| CBD-rich hemp oil | 0.90 |
| Ethylhexyl diphenyl phosphate | 8.5 |
| Tocopherol | 1.5 |
| Colorant | 0.1 |

The manufacturing method is similar to that of Example 6 above.

### Example 10: Soothing Collar

| Composition | Concentration (% p/p) |
|---|---|
| TPU | QS 100 |
| Isopropyl myristate | 3.00 |
| Eucalyptus citriodora | 0.75 |
| Peppermint essential oil | 0.75 |
| CBD-rich hemp oil | 0.90 |
| Ethylhexyl diphenyl phosphate | 8.5 |
| Dimethyl adipate, dimethyl | 10 |
| glutarate and dimethyl succinate | |
| Colorant | 0.5 |

The manufacturing method is similar to that of Example 5 above.

### Example 11: Soothing Collar

| Composition | Concentration (% p/p) |
|---|---|
| Squalane | 1 |
| CBD isolate | 0.75 |
| Peppermint essential oil | 0.75 |
| Ethylhexyl diphenyl phosphate | 15 |
| Dimethyl adipate, dimethyl | 13.5 |
| glutarate and dimethyl succinate | |
| Colorant | 0.1 |
| TPU | QS 100 |

The manufacturing method is similar to that of Example 5 above.
The stability results for the present device are as follows:

Temperature stability: 54° C.

| | D 0 | D 14 |
|---|---|---|
| Physical stability | No exudation, good | No exudation, good |
| | mechanical strength | mechanical strength |
| Chemical stability | 100% | 97% |
| (% CBD active | | |
| ingredient versus | | |
| initial amount | | |
| incorporated) | | |

UV light

| | D 0 | D 7 |
|---|---|---|
| Physical stability | No exudation, good | No exudation, good |
| | mechanical strength | mechanical strength |

The results show that the device is stable and allowed the required active ingredients, including CBD, to be incorporated effectively.

### Example 12: In Vitro Study of the Kinetics of CBD Release from Devices According to the Invention

An in vitro test was developed by the applicant to be able to verify that the active ingredient incorporated stably in the device according to the invention could also be effectively delivered over a period of time of up to at least four weeks, in order to treat the subject to which the device is applied.
The choice of a kinetic in oil simulates the contact of the device with the sebum of the skin or fur which allows the active ingredients to be released to treat the subject. Examples of collars for dogs previously produced according to the examples described above were tested. Some tests are still in progress on the filing date of the present application.
The operating method for this analysis for different examples of collars is as follows:
1) Remove the sheet wound around the collar to keep it rolled up before use.
2) Weigh and record the initial weight of the collar.
3) Place the collar in a 250 ml flask.
4) Add olive oil until the collar is completely immersed, and weigh.
5) Stir and keep stirring for the full time of the kinetic study.
6) At each time determined for analysis (D1, D3, D7, D10, D14, D21, D28, for example), take a sample of the oil.
7) Extract the CBD from the oil in this sample using an appropriate solvent.
8) Analyze the extract using HPLC to quantify the amount of CBD released into the medium. An amount is obtained in mg or as a percentage relative to the amount initially incorporated. The analysis is carried out using reversed-phase HPLC with external calibration from a certified CBD standard.
9) Plot the curve of CBD release in the oil over time.
   The results are obtained by averaging the values of three similar devices tested.

A) Results for the Release of CBD from a Collar According to Example 10:

| | CBD amount dosed in collars in mg |
|---|---|
| | (average of three collars) |
| Day | % release |
| 0 | 0% |
| 1 | 13% |
| 3 | 21% |
| 7 | 29% |
| 9 | 31% |
| 10 | 32% |
| 14 | 38% |
| 15 | 39% |
| 21 | 44% |
| 28 | 47% |

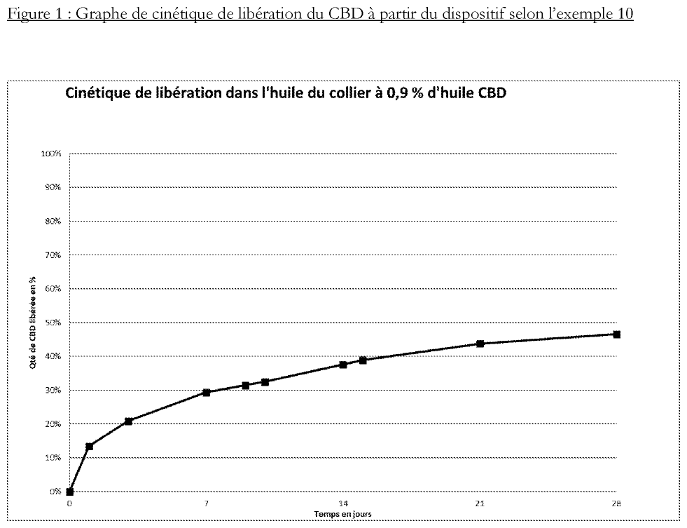

The device according to the invention in accordance with Example 10 in the form of a collar allowed the required active ingredients, including CBD, to be effectively and stably incorporated. The above results and the corresponding curve in FIG. 1 prove a gradual release of the CDB over time for the long-term treatment of the subject to be treated, such as a dog. At 28 days, it may be noted that a plateau has not been reached and that release continues.

B) Results for the Release of CBD from a Collar According to Example 5

| | CBD amount dosed in collars in mg |
|---|---|
| | (average of three collars) |
| Day | % release |
| 0 | 0% |
| 1 | 14% |
| 3 | 23% |
| 7 | 31% |
| 10 | 34% |
| 14 | In progress |
| 15 | In progress |
| 21 | In progress |
| 28 | In progress |

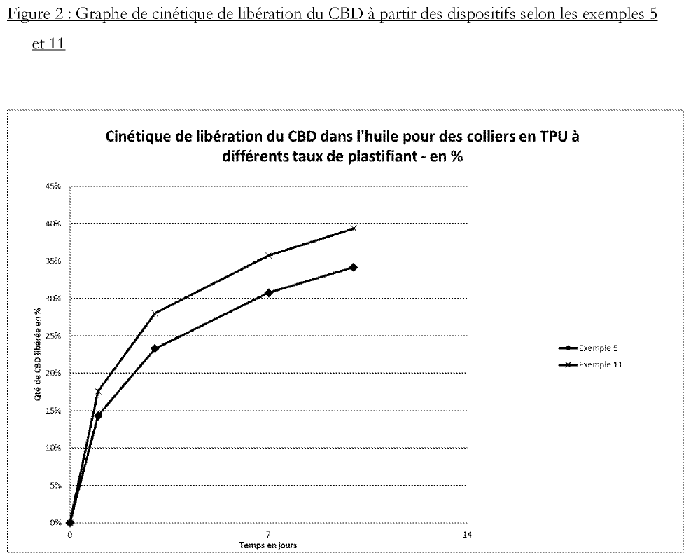

The device according to the invention in accordance with Example 5 in the form of a collar allowed the required active ingredients, including CBD, to be effectively incorporated. The above results and the corresponding curve in FIG. 2 prove a gradual release of the CDB over time. The tests in progress allow the release to be assessed over ten days. It should be understood that the test continues and that the slope of the curve allows easy extrapolation as to the continuing release over time.

C) Results for the Release of CBD from a Collar According to Example 11

| | CBD amount dosed in collars in mg |
|---|---|
| | (average of three collars) |
| Day | % release |
| 0 | 0% |
| 1 | 18% |
| 3 | 28% |
| 7 | 36% |
| 10 | 39% |
| 14 | In progress |
| 15 | In progress |
| 21 | In progress |
| 28 | In progress |

The device according to the invention in accordance with Example 11 in the form of a collar allowed the required active ingredients, including CBD, to be effectively incorporated. The above results and the corresponding curve in FIG. 2 prove a gradual release of the CDB over time. The tests in progress allow release to be assessed over ten days. It should be understood that the test continues and that the slope of the curve allows easy extrapolation as to the continuing release over time.
The curve in FIG. 2 also allows the release kinetics of the devices according to Examples 5 and 11 to be compared. These two devices according to the invention differ only in the amount of plasticizer in the composition.
These results show that the level of plasticizing compounds in the composition allows the active ingredient release kinetics for the devices according to the invention to be modulated.

1. A device for topical application formed of a single-layer polymer matrix characterized in that the polymer is chosen from the group consisting of an ethylene vinyl acetate copolymer and a polyurethane, said matrix also being characterized in that it comprises at least one cannabinoid derivate and between 0.5% and 20% by total weight of the matrix of an essential oil or a monoterpenic derivative of essential oils.

2. The device according to claim 1, characterized in that the polymer is non-adhesive.

3. The device according to claim 1, characterized in that the polymer is an ethylene vinyl acetate copolymer.

4. The device according to claim 1, characterized in that the polymer is a polyurethane.

5. The device according to claim 1, characterized in that the cannabinoid derivative is cannabidiol (CBD) free of delta-9-tetrahydrocannabinol (THC).

6. The device according to claim 1, characterized in that the essential oils or monoterpenic derivatives of essential oils are chosen from linalool, menthol or essential oils of lavender, lavandin, Italian helichrysum, cedar, lemon, citronella, carrot, ginger, niaouli, sweet orange, clove, Eucalyptus citriodora, Eucalyptus radiata, peppermint, geranium, valerian, Nepeta cataria, chamomile, ylang ylang, clary sage, mandarin, sandalwood, bergamot, marjoram, frankincense, geranium, thyme, juniper or mixtures thereof.

7. The device according to claim 1, characterized in that it comprises a vectorizing compound chosen from oils of organic origin, oils of synthetic origin, oils of mineral origin, oils of plant origin, esters such as succinate, glutarate and adipate mixtures, omega 3 polyunsaturated fatty acid oils, fatty acid esters, glycol ethers, squalene and squalene derivatives.

8. The device according to claim 1, characterized in that it comprises a plasticizing compound chosen from ethylhexyl diphenyl phosphate, dioctyl adipate, diisooctyl adipate, dibutyl adipate, dimethyl adipate, dimethyl glutarate, dimethyl succinate, or a mixture thereof.

9. The device according to claim 1, characterized in that it is presented in the form of a collar, bracelet, medallion or textile coating.

10. The device according to claim 1, characterized in that the cannabidiol is present in the form of a CBD isolate or a CBD oil.

11. A device for topical application in a thermoplastic polymer matrix, loaded with active ingredients for use in the relief of pain or stress in an animal, characterized in that said device is placed in contact with the skin and/or fur, and in that said polymer matrix comprises at least one non-adhesive thermoplastic polymer chosen from a polyurethane or an ethylene vinyl acetate copolymer, and in that the active ingredients comprise at least one cannabidiol isolate, free of delta-9-tetrahydrocannabinol or a cannabidiol oil, free of THC and between 0.5% and 20% p/p of at least one essential oil or a compound of an essential oil.

12. The device according to claim 11 for use in the relief of stress or muscular, joint or rheumatic pain, such as tendinitis, arthritis and arthrosis.

13. A method of manufacturing a device consisting of a thermoplastic polymer matrix comprising at least one non-adhesive thermoplastic polymer chosen from a polyurethane or an ethylene vinyl acetate copolymer, and in that the active ingredients comprise at least one cannabidiol isolate, free of delta-9-tetrahydrocannabinol or a cannabidiol oil free of delta-9-tetrahydrocannabinol and between 0.5% and 20% p/p of at least one essential oil or a compound of an essential oil, said method comprising the steps of heating the polymer to the chosen temperature, preparing the active phase, mixing the active phase with the polymer, then shaping the device, characterized in that said method does not comprise a step of adding specific excipients or particulate structures to help incorporate the active ingredients in the polymer matrix.

14. A product obtained by the method according to claim 13.

15. The device according to claim 2, characterized in that the polymer is an ethylene vinyl acetate copolymer.

16. The device according to claim 2, characterized in that the polymer is a polyurethane.

17. The device according to claim 2, characterized in that the cannabinoid derivative is cannabidiol (CBD) free of delta-9-tetrahydrocannabinol (THC).

18. The device according to claim 2, characterized in that the cannabidiol is present in the form of a CBD isolate or a CBD oil.

19. The device according to claim 2, characterized in that the essential oils or monoterpenic derivatives of essential oils are chosen from linalool, menthol or essential oils of lavender, lavandin, Italian helichrysum, cedar, lemon, citronella, carrot, ginger, niaouli, sweet orange, clove, Eucalyptus citriodora, Eucalyptus radiata, peppermint, geranium, valerian, Nepeta cataria, chamomile, ylang ylang, clary sage, mandarin, sandalwood, bergamot, marjoram, frankincense, geranium, thyme, juniper or mixtures thereof.

20. The device according to claim 2, characterized in that it comprises a vectorizing compound chosen from oils of organic origin, oils of synthetic origin, oils of mineral origin, oils of plant origin, esters such as succinate, glutarate and adipate mixtures, omega 3 polyunsaturated fatty acid oils, fatty acid esters, glycol ethers, squalene and squalene derivatives.
